SEQUENCE LISTING (3 sequences)

SEQ ID NO: 1 - protein, 9 aa, Homo sapiens
TEFTTALQR

SEQ ID NO: 2 - protein, 8 aa, Homo sapiens
DLGSELVR

SEQ ID NO: 3 - protein, 11 aa, Homo sapiens
DLIGFGLQVAK

(19) United States
(12) Patent Application Publication
(10) Pub. No.: US 2019/0234958 A1
(43) Pub. Date: Aug. 1, 2019

(54) Detection of Exon 14 Deletion in the Met Protein

(71) Applicant: NantOmics, LLC, Culver City, CA (US)

(72) Inventors: Todd A. HEMBROUGH, Gaithersburg, MD (US); Fabiola CECCHI, Potomac, MD (US)

(21) Appl. No.: 16/213,367

(22) Filed: Dec. 7, 2018

Related U.S. Application Data
(63) Continuation of application No. PCT/US17/36450, filed on Jun. 7, 2017.
(60) Provisional application No. 62/346,562, filed on Jun. 7, 2016.

(51) Int. Cl. G01N 33/68; G01N 1/30; G01N 33/574; A61K 31/4545; A61P 35/00

(52) U.S. Cl. CPC G01N 33/6848; G01N 1/30; G01N 2001/305; A61K 31/4545; A61P 35/00; G01N 33/574

(57) ABSTRACT

The current disclosure provides for specific peptides, and derived ionization characteristics of the peptides, from the Hepatocyte Growth Factor Receptor (Met) protein that are particularly advantageous for quantifying the Met protein directly in biological samples that have been fixed in formalin by the method of Selected Reaction Monitoring (SRM) mass spectrometry, or what can also be termed as Multiple Reaction Monitoring (MRM) mass spectrometry. Methods also are provided for detecting the presence of the Met (Ex14del) mutant protein.

### CROSS REFERENCE TO RELATED APPLICATIONS

This application claims priority under 35 U.S.C. § 119(e) from U.S. Provisional Patent Application Ser. No. 62/346,562, filed Jun. 7, 2016, the entire contents of which are hereby incorporated by reference.

### INTRODUCTION

A mass spectrometry-based SRM/MRM assay is described using three specific peptides derived from the amino acid sequence of the Hepatocyte Growth Factor Receptor protein, referred to herein as Met, and which is also referred to in the scientific literature as the HGF/SF receptor, proto-oncogene c-Met, cMet, scatter factor receptor, and tyrosine-protein kinase Met. The assay determines expression of a specific form of the Met protein in tissue, advantageously in formalin-fixed tissue. More specifically, the assay determines if the protein domain encoded by exon 14 of the MET gene is present or absent in the Met protein expressed in the tissue. This particular version of the Met protein, termed Met (Ex14del), appears in approximately 4% of all non-small cell lung cancers (NSCLC) and results from a mutation, or mutations, in the region of the genome that controls precise MET RNA splicing whereby the presence of one or more of these mutations leads to a skipping event of exon 14 during RNA processing, which in turn gives rise to the Met (Ex14del) protein. Loss of MET exon 14 leads to increased Met stability and prolonged signaling upon HGF stimulation, leading to increased growth and division of the cancer cells.

The peptide sequence and fragmentation/transition ions for each of the three specific peptides are used in a mass spectrometry-based Selected Reaction Monitoring (SRM) assay, which can also be referred to as a Multiple Reaction Monitoring (MRM) assay, and which is referred to herein as SRM/MRM. This SRM/MRM assay can be used to assess the expression and integrity of the Met protein, specifically to detect the presence or absence of the protein domain encoded by exon 14 of the MET gene in the expressed Met protein. The SRM/MRM assay can detect and measure the three specific peptides directly in complex protein lysate samples prepared from cells procured from patient tissue samples, such as formalin fixed cancer patient tissue.

Detecting expression of the Met (Ex14del) protein in tumor cells present within formalin fixed tumor tissue from a patient can inform the cancer treatment decision of the patient by indicating treatment with Met inhibitors such as crizotinib, tivantinib, cabozantinib, and foretinib that specifically inhibit the phosphorylation function of the Met protein.

### SUMMARY

Methods are provided for measuring the level of the Hepatocyte Growth Factor Receptor (Met) protein in a human biological sample of formalin-fixed tissue. The methods involve detecting and quantifying the amount of a first and a second Met fragment peptide in a protein digest, such as a protease digest, prepared from the human biological sample using mass spectrometry. The first Met fragment peptide is SEQ ID NO:1, and the second Met fragment peptide is SEQ ID NO:3. The methods may further include detecting and quantitating a Met fragment peptide having the sequence of SEQ ID NO:2. These amounts are used to calculate the level of Met protein in the sample, where the level is a relative or absolute level.

Methods also are provided for detecting the presence or absence of the Met (Ex14del) mutant protein in a human biological sample of formalin-fixed tissue. The methods involve detecting the presence or absence of a Met fragment peptide in a protein digest, such as a protease digest, prepared from the human biological sample using mass spectrometry; where the Met fragment peptide is SEQ ID NO:2. These methods may further involve detecting and quantitating the level of Met protein in the sample of formalin-fixed tissue, by detecting and quantifying the amount of a first Met fragment peptide having the sequence of SEQ ID NO:1, and a second Met fragment peptide having the sequence of SEQ ID NO:3, and calculating the level of Met protein in the sample, where the level is a relative level or an absolute level.

The sample used in the methods described above may optionally be paraffin embedded tissue and may be from a tumor. The protein digest may be fractionated prior to detecting and/or quantifying the amount of the Met fragment peptides.

The Met fragment peptides may be quantified by comparing an amount of the first and second Met fragment peptides in one biological sample to the amount of the same Met fragment peptides in a different and separate biological sample. In another embodiment, the Met fragment peptides may be quantified by determining the amount of the Met fragment peptides in a biological sample by comparison to added internal standard peptides of known amount, where the Met fragment peptides in the biological sample are compared to internal standard peptides having the same respective amino acid sequences, and where the internal standard peptides are isotopically labeled peptides.

Detecting and/or quantifying the amount of the Met fragment peptides and/or the presence of Met (Ex14del) in the protein digest may be used to indicate the presence of modified or unmodified Met protein and an association with cancer in the subject. The results of detecting and/or quantifying the amount of the Met fragment peptides, or the level of the Met protein, and/or the presence of Met (Ex14del) may be correlated to the diagnostic stage/grade/status of the cancer. This correlation may be combined with detecting and/or quantifying the amount of other proteins or peptides from other proteins in a multiplex format to provide additional information about the diagnostic stage/grade/status of the cancer.

The methods described above may further include comprising administering to the patient a therapeutically effective amount of a therapeutic agent, where the therapeutic agent and/or amount of the therapeutic agent administered is based upon amount of the Met fragment peptides or the level of Met protein and/or the presence of Met (Ex14del). Advantageously, the therapeutic agent binds the Met protein and/or inhibits its biological activity. The therapeutic agent may be, for example, crizotinib, tivantinib, cabozantinib, or foretinib, or combinations thereof.

### BRIEF DESCRIPTION OF THE DRAWINGS

FIG. 1 shows that 3 peptides (SEQ ID NO:1, SEQ ID NO:2, and SEQ ID NO:3) were positively detected by visualization of the reproducible mass spectrometry peaks, indicating the presence of all 3 peptides in the normal version of Met protein. This shows positive detection and quantitation of the Met protein domain encoded by exon 14 of the MET gene. The FIGURE also shows no visual detection (Not Detected) of positive mass spectrometry peaks for SEQ ID NO:2 in H596 and Hs746T cell lines, further indicating expression of the Met protein that does not contain the protein domain encoded by exon 14 of the MET gene in these cell lines and the ability to detect expression of the Met (Ex14 del) protein.

### DETAILED DESCRIPTION

The assay described herein detects the presence of three different peptides of the human Met protein using SRM/MRM methodology. The first peptide resides within the extracellular domain of the Met protein, and the second and third peptides reside within the intracellular domain of the Met protein. The second peptide is a fragment of the Met protein segment encoded by Exon 14 (amino acids 963-1010) of the MET gene. The presence of these three peptides in the expressed Met protein is determined relative one to another within the same sample. This assay can be used to analyze both the expression levels of the Met protein and the integrity of the expressed form of the Met protein—the presence of the extracellular and intracellular domains of the expressed Met protein can be determined, as can the presence (or absence) within the full length Met protein of the intracellular domain encoded by exon 14 (amino acids 963-1010) of the MET gene.

SRM/MRM methodology detects and quantitates a specific peptide by detecting a unique signature peak of that peptide in a biological sample and comparing the peak area to the SRM/MRM signature peak area of an internal standard peptide spiked into the same biological sample. In one embodiment, each internal standard is a synthetic version of one of the three Met peptides, having the exact same amino acid sequence but containing one or more amino acid residues labeled with one or more heavy isotopes. Each isotope labeled internal standard is synthesized so that when analyzed by mass spectrometry it generates a predictable and consistent SRM/MRM signature peak that is different and distinct from the native Met peptide signature peak and which can be used as a comparator peak. When the internal standard is spiked in known amounts into a protein preparation from a biological sample and analyzed by mass spectrometry, the SRM/MRM signature peak area of the native peptide is compared to the SRM/MRM signature peak area of the internal standard peptide, and this numerical comparison indicates either the absolute molarity and/or absolute weight of the native peptide present in the original protein preparation from the biological sample. Detection and quantitation of specific Met peptides are displayed according to the amount of protein analyzed per sample. SRM/MRM peptide detection and quantification can be performed across many Met peptides simultaneously in a single sample thus making possible the analysis of all three Met peptides in the SRM/MRM assay.

The SRM/MRM assay method can be used to aid diagnosis of cancer, for example, directly in patient-derived tissue, such as formalin fixed tissue, and to aid in determining which therapeutic agent would be most advantageous for use in treating that patient. Cancer tissue that is removed from a patient either through surgery, such as for therapeutic removal of partial or entire tumors, or through biopsy procedures conducted to determine the presence or absence of suspected disease, is analyzed to determine whether or not Met, and/or other proteins, and which forms of proteins, are present in that patient tissue. Moreover, the expression level of a protein, or multiple proteins, can also be determined. Assays of protein levels and/or various forms and isoforms of proteins (e.g., Met Exon 14 deletion protein) can also be used to inform a treatment strategy. Once the Met protein expression characteristics of the cancer have been determined, that information informs physicians about which therapeutic agents (chemical and biological) will most likely have the greatest chance of helping the patient. Matching information from a Met protein assay to potential therapeutic agents helps to inform a personalized medicine approach to treating cancer in a patient whose Met protein may or may not contain the protein domain encoded by exon 14 of the MET gene.

In principle, any predicted peptide derived from the Met protein, prepared for example by digesting with a protease of known specificity (e.g. trypsin), can be used as a surrogate reporter to detect expression of the Met protein in a sample using a mass spectrometry-based SRM/MRM assay. In practice, however, the identity of the peptide or peptides (if any) that can be used to analyze the Met protein is highly unpredictable. This is especially the case in formalin-fixed tissue, where protein crosslinking leads to further increases in unpredictability.

Suitable tissue digests containing Met fragment peptides may be generated by a variety of means including by the use of the Liquid Tissue protocol provided in U.S. Pat. No. 7,473,532. The Liquid Tissue protocol and reagents are capable of producing peptide samples suitable for mass spectroscopic analysis from formalin fixed paraffin embedded tissue by proteolytic digestion of the proteins in the tissue/biological sample. In the Liquid Tissue protocol the tissue/biological is heated in a buffer for an extended period of time (e.g., from about 80° C. to about 100° C. for a period of time from about 10 minutes to about 4 hours) to reverse or release protein cross-linking. The buffer employed is a neutral buffer, (e.g., a Tris-based buffer, or a buffer containing a detergent). Following heat treatment the tissue/biological sample is treated with one or more proteases, including but not limited to trypsin, chymotrypsin, pepsin, and endoproteinase Lys-C for a time sufficient to disrupt the tissue and cellular structure of said biological sample and to liquefy said sample (e.g., a period of time from 30 minutes to 24 hours at a temperature from 37° C. to 65° C.). The result of the heating and proteolysis is a liquid, soluble, dilutable biomolecule lysate.

The most widely and advantageously available form of tissue from cancer patient tissue is formalin fixed, paraffin embedded tissue. Formaldehyde/formalin fixation of surgically removed tissue is by far the most common method of preserving cancer tissue samples worldwide and is the accepted convention for standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods that analyze formalin fixed cancer tissue are the most accepted and heavily utilized methods for analysis of cancer patient tissue.

Met peptides utilized in this SRM/MRM assay (having the amino acid sequences shown in Tables 1 and 2) were derived from the Met protein by protease digestion of all the proteins within a complex Liquid Tissue lysate prepared from cells procured from formalin fixed cancer tissue. Unless noted otherwise, in each instance the protease was trypsin. The Liquid Tissue lysate was then analyzed by mass spectrometry to determine which, if any, peptides derived from the Met protein could be efficiently and reproducibly detected and analyzed by mass spectrometry for determining (a) expression of the Met protein and (b) whether or not the coding domain from exon 14 of the MET gene is present in the expressed Met protein.

Identification of the specific three peptides for mass-spectrometric analysis to define this SRM/MRM assay is based on: (1) ability to detect the extracellular, intracellular, and exon 14 protein domains within the expressed Met protein; (2) experimental determination of which peptide or peptides from these three domains of the Met protein ionize in mass spectrometry analyses of Liquid Tissue lysates; and (3) the ability of the peptides to survive the protocol and experimental conditions used in preparing a Liquid Tissue lysate.

The assay described here detects the presence or absence of a variant of the Met protein that, when expressed in tumor cells, renders the tumor cells susceptible to treatment with Met protein inhibitor molecules. Rare mutations/deletions in the MET gene found at the splice donor or acceptor sites around or involving MET exon 14 in NSCLC are known as MET exon 14 deletion (METex14) mutations. When present in the genome of tumor cells these mutations/deletions result in defective Met messenger RNA (mRNA) splicing, leading to a defective Met protein that does not contain the protein coding region of exon 14 of the MET gene. Initially reported in both small cell lung cancer in 2003 and then in non-small cell lung cancer (NSCLC) in 2005, the significance of these splice site mutations/deletions was demonstrated in 2006 where multiple point mutations and deletions in the splice donor and acceptor sites resulted in the exon 14 of MET gene being spliced out of the eventual mature MET mRNA. The portion of the protein encoded by exon 14 is required for efficient recruitment of the ubiquitin ligase CBL, which targets Met for ubiquitin-mediated degradation. Specifically, CBL targets tyrosine residue 1003 (Y1003) where ubiquitin is attached to the tyrosine residue and leads to lysosomal degradation of the Met protein. Hence, “skipping” of the protein region that is encoded by MET exon 14 results in a Met protein that is less efficiently degraded, leading to a relative over-expression of Met protein with markedly enhanced Met activation (phosphorylation) and subsequent oncogenesis.

The Cancer Genome Atlas (TCGA) project estimates that the incidence of METex14 in adenocarcinoma is around 3-4%. Recently it has been found that cancers containing METex14 mutations re therapeutically “actionable” by inhibition using the class of Met tyrosine kinase inhibitors such as crizotinib, tivantinib, cabozantinib, and foretinib, resulting results in clinical benefit in NSCLC patients harboring such MET exon 14 alterations.

The three Met tryptic peptides that are used in the presently described SRM/MRM assay that analyzes expression of the Met protein by detecting the presence of the extracellular, intracellular, and the exon 14 coding domain of the Met protein are listed in Table 1. SEQ ID NO:1 resides in the Met extracellular domain, SEQ ID NO:2 resides in the intracellular domain of the Met protein and is found within the domain encoded by exon 14 of the MET gene, and SEQ ID NO:3 resides in the Met intracellular domain. The Met tryptic peptides listed in Table 1 are efficiently and reproducibly detected in Liquid Tissue lysates prepared from multiple types of cancer including breast, colon, and lung cancer. Each of those peptides is also effectively used for a quantitative SRM/MRM assay of the Met protein in Liquid Tissue lysates prepared from formalin fixed tissue. Thus, each of these peptides is suitable for conducting the presently described SRM/MRM assay of the Met protein on a Liquid Tissue lysate from any formalin fixed tissue originating from any biological sample or from any organ site in the body, and whereby all three peptides are assayed simultaneously in a single mass spectrometry assay.

TABLE 1

| | Peptide |
|---|---|
| Peptide | Sequence |
| SEQ ID NO: 1 | TEFTTALQR |
| SEQ ID NO: 2 | DLGSELVR |
| SEQ ID NO: 3 | DLIGFGLQVAK |

One consideration for conducting an SRM/MRM assay such as the presently described SRM/MRM assay is the type of instrument that may be employed in the analysis of the peptides. Although SRM/MRM assays can be developed and performed on any type of mass spectrometer, including a MALDI, ion trap, triple quadrupole, or ion trap/triple quadrupole hybrid, the most advantageous instrument platform for SRM/MRM assay is presently considered to be a triple quadrupole instrument platform. This type of a mass spectrometer is presently the most suitable instrument for analyzing a single isolated target peptide within a very complex protein lysate that may consist of hundreds of thousands to millions of individual peptides from all the proteins contained within a cell.

In order to most efficiently implement SRM/MRM assays for each of the three peptides derived from the Met protein it is desirable to utilize information in addition to the peptide sequence in the analysis. That additional information may be used in directing and instructing the mass spectrometer (e.g. a triple quadrupole mass spectrometer), to perform the correct and focused analysis of specific targeted peptide(s), such that the assay may be effectively performed. This additional information about target peptides in general, and about these three specific Met peptides in particular, may include one or more of the mono isotopic mass of the peptide, its precursor charge state, the precursor m/z value, the m/z transition ions, and the ion type of each transition ion. Additional peptide information used to perform the presently described SRM/MRM assay for the Met protein for all three of the Met peptides is shown in Table 2.

TABLE 2

| | | Mono | Precursor | | | |
|---|---|---|---|---|---|---|
| | | Isotopic | Charge | Precursor | Transition | Ion |
| SEQ ID | Peptide sequence | Mass | State | m/z | m/z | Type |
| SEQ ID | TEFTTALQR | 1065.5454 | 2 | 533.784 | 588.346 | y5 |
| NO: 1 | | | 2 | 533.784 | 689.394 | y6 |
| | | | 2 | 533.784 | 836.462 | y7 |
| SEQ ID | DLGSELVR | 887.4712 | 2 | 442.784 | 387.27 | y3 |
| NO: 2 | | | 2 | 442.784 | 435.737 | b8 + 2 |
| | | | 2 | 442.784 | 516.313 | y4 |
| | | | 2 | 442.784 | 603.345 | y5 |
| | | | 2 | 442.784 | 660.366 | y6 |
| SEQ ID | DLIGFGLQVAK | 1159.66 | 2 | 580.837 | 229.117 | b2 |
| NO: 3 | | | 2 | 580.837 | 615.381 | y6 |
| | | | 2 | 580.837 | 762.45 | y7 |
| | | | 2 | 580.837 | 819.471 | y8 |
| | | | 2 | 580.837 | 932.555 | y9 |

Assessment of Met protein levels and the presence/absence of the Met protein domain encoded by exon 14 of the MET gene in tissues based on analysis of formalin fixed patient-derived tissue can provide diagnostic, prognostic, and therapeutically-relevant information about each particular patient. In one embodiment, the presently described SRM/MRM assay provides methods for measuring the level of the Met protein in a biological sample, comprising detecting and quantifying the amount of two specified Met fragment peptides (SEQ ID NO:1 and SEQ ID NO:3) in a protein digest prepared from the biological sample using mass spectrometry; and calculating the level of Met protein in the sample; where level is a relative level or an absolute level. In a second embodiment, the presently described SRM/MRM assay involves detecting a specific Met fragment peptide (SEQ ID NO:2) that resides within the protein domain encoded by exon 14 of the MET gene. All three Met fragment peptides may be detected and quantitated in a biological sample by comparison to added internal standard peptides of known amount, wherein each of the SEQ ID NO:1, SEQ ID NO:2, and SEQ ID NO:3 Met fragment peptides in the biological sample is compared to three internal standard peptides respectively having the same amino acid sequence as SEQ ID NO:1, SEQ ID NO:2, and SEQ ID NO:3. In some embodiments the internal standard is an isotopically labeled internal standard peptide comprises one or more heavy stable isotopes selected from ¹⁸O, ¹⁷O, ³⁴S, ¹⁵N, ¹³C, ²H or combinations thereof.

The method described below was used to: (1) identify candidate peptides from the Met protein that can be used for the presently described mass spectrometry-based SRM/MRM assay for the Met protein, (2) develop individual SRM/MRM assays for each of the presently described peptides from the Met protein (SEQ ID NO:1, SEQ ID NO:2, and SEQ ID NO:3) and (3) develop a single multiplex SRM/MRM that simultaneously detects and quantifies each of the peptides in a Liquid Tissue lysate in order to determine expression of the Met protein and the presence or absence of the protein domain encoded by exon 14 of the MET gene in order to inform choice of optimal cancer therapy.

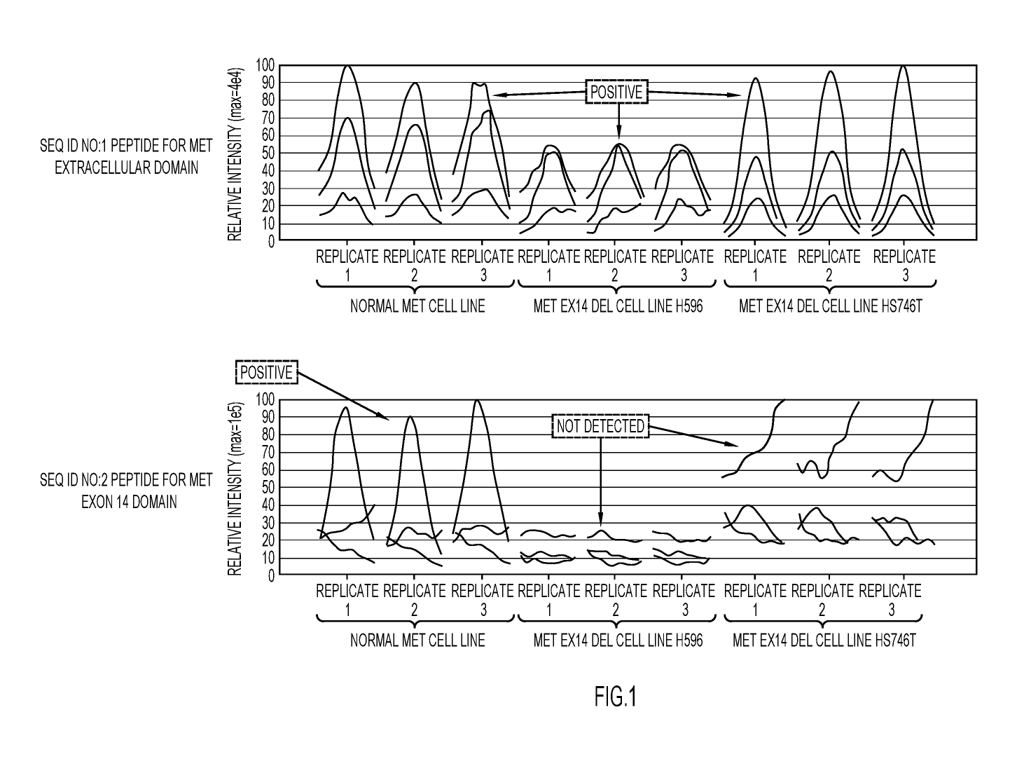

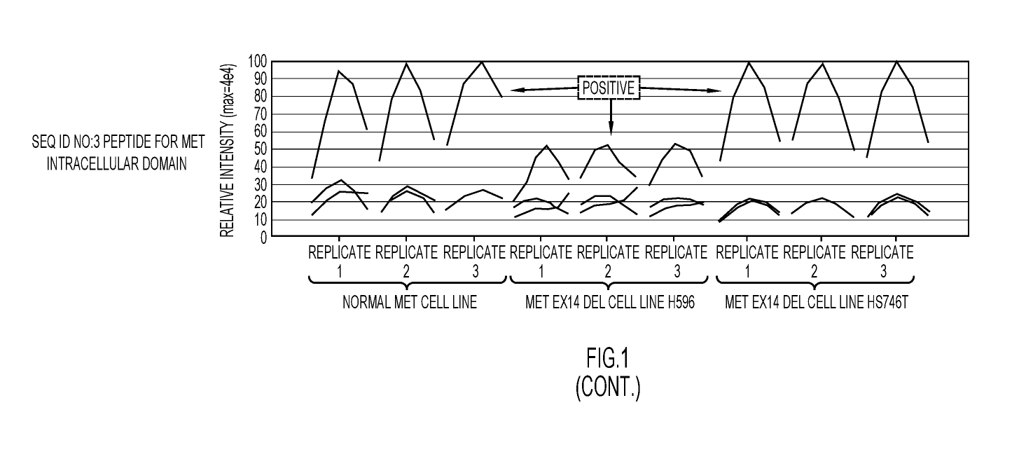

Experimental demonstration of the assay is shown in Table 3 and FIG. 1. Table 3 shows detection and quantitative SRM/MRM analysis, in triplicate, of all 3 presently described peptides in 3 Liquid Tissue lysates prepared from 3 different formalin fixed human cell lines, where the results are shown in units of amol/ug total protein analyzed. The first cell line, H226, is known to express the normal form of the Met protein that contains the exon 14 domain of the MET gene. SRM/MRM analysis of this cell line that expresses the normal version of the Met protein detected and quantitated all 3 peptides (SEQ ID NO:1, SEQ ID NO:2, and SEQ ID NO:3) where SEQ ID NO:1=694.50 amol/ug, SEQ ID NO:2=1101 amol/ug, SEQ ID NO:3=877 amol/ug. As demonstrated in FIG. 1, all 3 peptides (SEQ ID NO:1, SEQ ID NO:2, and SEQ ID NO:3) were positively detected by visualization of the reproducible mass spectrometry peaks thus indicating the presence of all 3 peptides in the normal version of Met protein. This indicates positive detection and quantitation of the Met protein domain encoded by exon 14 of the MET gene.

The two other cell lines in this experimental demonstration, H596 and Hs746T, are known to contain mutations around exon 14 splice junctions of the MET gene resulting in expression of the Met (Ex14del) version of the Met protein. Table 3 shows detection and quantitation of only 2 of these 3 peptides (SEQ ID NO:1 and SEQ ID NO:3) in both of these cell lines known to express the Met (Ex14del) version of the Met protein. Results indicate SEQ ID NO:1 was detected and quantitated in H596 at 493.53 amol/ug while and SEQ ID NO:3 was detected and quantitated in H596 at 636.33 amol/ug. Likewise, SEQ ID NO:1 was detected and quantitated in Hs746T at 3288.67 amol/ug while and SEQ ID NO:3 was detected and quantitated in Hs746T at 4063.17 amol/ug. FIG. 1 demonstrates positive detection of these peptides in these cell lines by visualization of the mass spectrometry peaks.

However, SEQ ID NO:2 was not detected in the H596 and Hs746T cell lines and could not be quantitated, indicating expression of the Met(Ex14 del) protein and demonstrating the ability of the assay to detect expression of this form of the Met protein in Liquid Tissue lysates from formalin fixed cells. FIG. 1 indicates no visual detection (Not Detected) of positive mass spectrometry peaks for SEQ ID NO:2 in H596 and Hs746T cell lines further indicating expression of the Met protein that does not contain the protein domain encoded by exon 14 of the MET gene in these cell lines and the ability to detect expression of the Met (Ex14 del) protein.

TABLE 3

| | | SEQ ID NO: 1 | SEQ ID NO: 2 | SEQ ID NO: 3 |
|---|---|---|---|---|
| Cell Line | Met Protein Status | Extracellular Domain | Exon 14 Domain | Intracellular Domain |
| H226 | Normal Met cell line | 694.50 amol/ug | 1101 amol/ug | 877 amol/ug |
| H596 | MET Ex14 del cell line 1 | 493.53 amol/ug | Not Detected | 636.33 amol/ug |
| Hs746T | MET Ex14 del cell line 2 | 3288.67 amol/ug | Not Detected | 4063.17 amol/ug |

### Assay Method

1. Identification of SRM/MRM candidate fragment peptides for the Met protein
   a. A Liquid Tissue protein lysate from a formalin fixed biological sample using a protease or proteases, (in this specific case, trypsin), was used to digest proteins
   b. All protein fragments in the Liquid Tissue lysate were analyzed on an ion trap tandem mass spectrometer and all fragment peptides from the Met protein were identified, where individual fragment peptides do not contain peptide modifications such as phosphorylations or glycosylations
   c. Preferred peptides used for development of the SRM/MRM assay were those identified by mass spectrometry directly in a complex Liquid Tissue protein lysate prepared from a formalin fixed biological sample
2. Mass Spectrometry Assay for Fragment Peptides from Met Protein
   a. SRM/MRM assay on a triple quadrupole mass spectrometer for three individual fragment peptides
      i. Assays were developed for one peptide that resides in the extracellular domain and two that reside within the intracellular domain of the Met protein, and further where one of the intracellular domain peptides resides within the portion of the Met protein encoded by exon 14 of the MET gene as identified in a Liquid Tissue lysate is applied to peptides from the Met protein
      ii. Optimal retention time for each fragment peptide was determined for optimal chromatography conditions including but not limited to, liquid chromatography, nano-reversed phase liquid chromatography, high performance liquid chromatography, and/or reverse phase high performance liquid chromatography
      iii. The mono isotopic mass of the peptide was determined, the precursor charge state for each peptide, the precursor m/z value for each peptide, the m/z transition ions for each peptide, and the ion type of each transition ion for each fragment peptide.
      iv. SRM/MRM assays were then conducted using the information from (i), (ii), and (iii) above on a triple quadrupole mass spectrometer where each peptide has a characteristic and unique SRM/MRM signature peak that precisely defines the unique SRM/MRM assay as performed on a triple quadrupole mass spectrometer
   b. SRM/MRM analysis was performed so that the amount of the fragment peptides of the Met protein was detected as a function of the unique SRM/MRM signature peak area, indicating both the relative and absolute amount of the protein in a particular protein lysate.
      i. Relative quantitation can be achieved by:
         1. Determining increased or decreased presence of the Met protein by comparing the SRM/MRM signature peak area from a given Met peptide detected in a Liquid Tissue lysate from one formalin fixed biological sample to the same SRM/MRM signature peak area of the same Met fragment peptide in at least a second, third, fourth or more Liquid Tissue lysates from least a second, third, fourth or more formalin fixed biological samples
         2. Determining increased or decreased presence of the Met protein by comparing the SRM/MRM signature peak area from a given Met peptide detected in a Liquid Tissue lysate from one formalin fixed biological sample to SRM/MRM signature peak areas developed from fragment peptides from other proteins, in other samples derived from different and separate biological sources, where the SRM/MRM signature peak area comparison between the 2 samples for a peptide fragment are normalized to amount of protein analyzed in each sample.
         3. Determining increased or decreased presence of the Met protein by comparing the SRM/MRM signature peak area for a given Met peptide to the SRM/MRM signature peak areas from other fragment peptides derived from different proteins within the same Liquid Tissue lysate from the formalin fixed biological sample in order to normalize changing levels of Met protein to levels of other proteins that do not change their levels of expression under various cellular conditions.
      ii. Absolute quantitation of a given peptide was achieved by comparing the SRM/MRM signature peak area for a given fragment peptide from the Met protein in an individual biological sample to the SRM/MRM signature peak area of an internal fragment peptide standard spiked into the protein lysate from the biological sample
         1. The internal standard is a labeled synthetic version of the fragment peptide from the Met protein that is being interrogated. This standard is spiked into a sample in known amounts, and the SRM/MRM signature peak area can be determined for both the internal fragment peptide standard and the native fragment peptide in the biological sample separately, followed by comparison of both peak areas
         2. This was applied to all three chosen Met fragment peptides
3. Fragment Peptide Detection and Quantitation to Cancer Diagnosis and Treatment
   a. Relative and/or absolute quantitation of all three fragment peptides of the Met protein was performed, demonstrating that correlation of the Met (Ex14del) expression to the status of cancer in patient tumor tissue is confirmed
   b. Correlation of Met (Ex14del) protein expression with clinical outcomes from different treatment strategies can be demonstrated, where this correlation has already been demonstrated in the field or can be demonstrated in the future through correlation studies across cohorts of patients and tissue from those patients. The assay method can be used to determine optimal treatment strategy.

1. A method for measuring a level of the Hepatocyte Growth Factor Receptor (Met) protein in a human biological sample of formalin-fixed tissue, the method comprising
detecting and quantifying an amount of a first and a second Met fragment peptide in a protein digest prepared from the human biological sample of formalin-fixed tissue using mass spectrometry; wherein the first Met fragment peptide is SEQ ID NO:1, and the second Met fragment peptide is SEQ ID NO:3, and
calculating the level of Met protein in the human biological sample of formalin-fixed tissue.

2. The method of claim 1, further comprising fractionating the protein digest prior to detecting and quantifying the amount of said Met fragment peptides.

3. (canceled)

4. The method of claim 1, wherein the formalin-fixed tissue is paraffin embedded formalin-fixed tissue.

5. The method of claim 1, wherein the formalin-fixed tissue is obtained from a tumor.

6. The method of claim 1, wherein quantifying said Met fragment peptides comprises comparing an amount of the first and the second Met fragment peptides in one biological sample to an amount of the same Met fragment peptides in a different and separate biological sample.

7. The method of claim 1, wherein quantifying said Met fragment peptides comprises determining the amount of the Met fragment peptides in the human biological sample of formalin-fixed tissue by comparison to added internal standard peptides of known amount, wherein said Met fragment peptides in the human biological sample of formalin-fixed tissue are compared to internal standard peptides having the same respective amino acid sequences, and wherein the internal standard peptides are isotopically labeled peptides.

8. The method of claim 1, wherein detecting and quantifying the amount of said Met fragment peptides in the protein digest indicates the presence of modified or unmodified Met protein and an association with cancer.

9. The method of claim 8, further comprising correlating the results of the detecting and quantifying the amount of said Met fragment peptides, or the level of said Met protein to the diagnostic stage/grade/status of the cancer.

10. The method of claim 9, wherein correlating the results of the detecting and/or quantifying the amount of said Met fragment peptides, or the level of said Met protein to the diagnostic stage/grade/status of the cancer is combined with detecting and/or quantifying an amount of other proteins or peptides from other proteins in a multiplex format to provide additional information about the diagnostic stage/grade/status of the cancer.

11. The method of claim 1, further comprising administering to the patient from which said biological sample was obtained a therapeutically effective amount of a therapeutic agent, wherein the therapeutic agent and/or amount of the therapeutic agent administered is based upon the amount of said Met fragment peptides or the level of Met protein.

12. (canceled)

13. The method of claim 1 further comprising detecting and quantitating a Met fragment peptide having the sequence of SEQ ID NO:2.

14. A method of detecting the presence or absence of Met (Ex14del) mutant protein in a human biological sample of formalin-fixed tissue, the method comprising detecting the presence or absence of a Met fragment peptide in a protein digest prepared from the human biological sample of formalin-fixed tissue using mass spectrometry; wherein said Met fragment peptide is SEQ ID NO:2.

15. The method of claim 14, further comprising detecting and quantitating the level of Met protein in the human biological sample of formalin-fixed tissue; by detecting and quantifying the amount of a first Met fragment peptide having the sequence of SEQ ID NO:1, and a second Met fragment peptide having the sequence of SEQ ID NO:3, and calculating the level of Met protein in the sample.

16. The method of claim 14, further comprising the step of fractionating said protein digest prior to detecting and/or quantifying the amount of said Met fragment peptides.

17. (canceled)

18. The method of claim 14, wherein the formalin-fixed tissue is paraffin embedded tissue.

19. The method of claim 14, wherein the formalin-fixed tissue is obtained from a tumor.

20. The method of claim 14, wherein quantifying the Met fragment peptides comprises comparing an amount of the Met fragment peptides in one biological sample to an amount of the same Met fragment peptides in a different and separate biological sample.

21. The method of claim 14, wherein quantifying said Met fragment peptides comprises determining the amount of the Met fragment peptides in the human biological sample of formalin-fixed tissue by comparison to added internal standard peptides of known amount, wherein said Met fragment peptides in the human biological sample of formalin-fixed tissue are compared to internal standard peptides having the same respective amino acid sequences, and wherein the internal standard peptides are isotopically labeled peptides.

22. The method of claim 14, wherein detecting and/or quantifying the amount of said Met fragment peptides and/or the presence of Met (Ex14del) in the protein digest indicates the presence of Met protein and/or Met (Ex14del) protein and an association with cancer.

23. The method of claim 22, further comprising correlating the results of the detecting and quantifying the amount of said Met fragment peptides, or the level of said Met protein and/or the Met (Ex14 del) protein to the diagnostic stage/grade/status of the cancer.

24. The method of claim 23, wherein correlating the results of the detecting and/or quantifying the amount of the Met fragment peptides, or the level of said Met protein and/or said Met (Ex14 del) protein to the diagnostic stage/grade/status of the cancer is combined with detecting and/or quantifying an amount of other proteins or peptides from other proteins in a multiplex format to provide additional information about the diagnostic stage/grade/status of the cancer.

25. The method of claim 14, further comprising administering to a patient from which the human biological sample of formalin-fixed tissue was obtained a therapeutically effective amount of a therapeutic agent, wherein the therapeutic agent and/or amount of the therapeutic agent administered is based upon the amount of the Met fragment peptides or the level of Met protein.

26. The method of claim 25, wherein the therapeutic agent binds the Met protein and/or inhibit its biological activity.

27. The method of claim 26, wherein the therapeutic agent is selected from the group consisting of crizotinib, tivantinib, cabozantinib, and foretinib.
